SEQUENCE LISTING (2 sequences)

SEQ ID NO: 1 - DNA, 23 bp, Artificial sequence
  Synthetic construct
graacccactgcttaassctcaa

SEQ ID NO: 2 - DNA, 23 bp, Artificial sequence
  Synthetic construct
tgttcgggcgccactgctagaga

(19) United States
(12) Patent Application Publication
(10) Pub. No.: US 2023/0285442 A1
(43) Pub. Date: Sep. 14, 2023

(54) USE OF OLIGOCHITOSANS AND DERIVATIVES THEREOF FOR NEUTRALIZING VIRAL AGENTS

(71) Applicants: Subhra MOHAPATRA, Lutz, FL (US); Shyam S. MOHAPATRA, Lutz, FL (US); Julio GARAY, Tampa, FL (US); Mazen HANNA, Tampa, FL (US); Karthick MAYILSAMY, Tampa, FL (US)

(72) Inventors: Subhra MOHAPATRA, Lutz, FL (US); Shyam S. MOHAPATRA, Lutz, FL (US); Julio GARAY, Tampa, FL (US); Mazen HANNA, Tampa, FL (US); Karthick MAYILSAMY, Tampa, FL (US)

(21) Appl. No.: 17/914,225

(22) PCT Filed: Mar. 26, 2021
(86) PCT No.: PCT/US2021/024266

§ 371 Date: Sep. 23, 2022

Related U.S. Application Data
(60) Provisional application No. 63/000,392, filed on Mar. 26, 2020.

(51) Int. Cl. A61K 31/722; A61P 31/12

(52) U.S. Cl. CPC A61K 31/722; A61P 31/12

(57) ABSTRACT

Described herein are methods for neutralizing a virus. The methods involve interacting the virus with an oligochitosan or a derivative thereof having a molecular weight of at least 1 KDa and a degree of deacetylation of at least 1%. The methods described herein have numems applications with respect to the treatment or prevention of viral infections caused by a human immunodeficiency virus, a paramyxovirus, an orthomyxovirus, a coronavirus, or a filovirus as well as the detection and quantification of viral load. Also described herein are the synthesis of the oligochitosan and derivatives thereof.

### CROSS-REFERENCE TO RELATED APPLICATIONS

This application claims the benefit of and priority to co-pending U.S. Provisional Patent Application No. 63/000,392, filed on Mar. 26, 2020, the contents of which are incorporated by reference herein in their entireties.

### CROSS REFERENCE TO SEQUENCE LISTING

The genetic components described herein are referred to by sequence identifier numbers (SEQ ID NO). The SEQ ID NOs correspond numerically to the sequence identifiers <400>1, <400>2, etc. The Sequence Listing, in written computer readable format (CRF), is incorporated by reference in its entirety.

### BACKGROUND

There is an ongoing effort to develop new compounds and methodologies for the treatment and prevention of viral infections. RNA viruses such as retroviruses pose a significant public health threat. Examples of such viruses include, but are not limited to, HIV, paramyxoviruses, orthomyxoviruses, coronaviruses, and filoviruses.

The recent emergence of Severe Acute Respiratory Syndrome Virus-2 (SARS-CoV2, synonym CoV2) in December of 2019 in Wuhan, China and the subsequent declaration of a pandemic by the World Health Organization, reaffirms the clinical significance of emerging coronaviruses. Given that cross-reactive immunity from other viral exposure is unlikely, it indicates that the vast majority of people could be susceptible to infection (Park S E. Clin Exp Pediatr. 2020. Epub Mar. 7, 2020. doi: 10.3345/cep.2020.00493. PMID: 32252141.1). CoV2 has been characterized as a virus causing pneumonia and severe respiratory distress. These severe manifestations of viral infection, however, seem to particularly burden the elderly and those with underlying conditions, however, severe cases have been seen in the young as well. Along with SARS-CoV epidemic in 2003 and MERS-CoV in 2012, CoV2 has become the third coronavirus to reach epidemic status, and subsequently pandemic status in the last two decades. Though coronaviruses have been human pathogens since 1960s, this surge in novel coronaviruses that have a significant impact on human health has led to the dire need for increased methods for rapid and reliable detection, prevention, treatment, and characterization of coronaviruses. It has been suggested that CoV2 will continue to remain as a serious agent and add to the repertoire of other seasonal respiratory infections caused by influenza, respiratory syncytial virus and rhinovirus. Further, despite significant efforts made during the pandemic, about one-third of the US population does not practice adequate public health measures such as face masking and about one-third the population avoid vaccination. Hence, there is a dire need to develop additional broad anti-viral agents against respiratory viral infections.

One of the unique features of CoV2 is that it is easily transmitted compared to other viruses (CoV2 R₀=2.2 R₀=0.3 for Influenza). Also, individuals infected with CoV2 remain asymptomatic and yet pass on the virus to family, friends and colleagues at work, thus spreading the infection in the population (Rothe C et al, N Engl J Med. 2020; 382(10):970-1. Epub Feb. 1, 2020. doi: 10.1056/NEJMc2001468. PMID: 32003551). In the absence of widespread testing, the ability of this virus to be transmitted from asymptomatic carriers is considered an underlying factor for this coronavirus's unprecedented spread across the globe (Lai CC et al, J Microbiol Immunol Infect. 2020. Epub Mar. 17, 2020. doi: 10.1016/j.jmii.2020.02.012. PubMed PMID: 32173241). Currently, CoV2 infections are treated with antivirals such as remdesivir (GS5743), an adenosine analog approved for Ebola virus with an ssRNA genome (Sheahan T P et al Science translational medicine. 2017; 9(396). Epub Jul. 1, 2017. doi: 10.1126/scitranslmed.aa13653. PMID: 28659436), or an anti-inflammatory dexamethasone. However, these drugs have moderate efficacy and must be given early in the infection. Hence there is an urgent dire need to develop pre and post-exposure prophylaxis, which can help decrease the virus expansion in the lung prior to symptoms in those identified by contact tracing and after being identified as positive post-exposure.

### SUMMARY

Described herein are methods for neutralizing a virus. The methods involve interacting the virus with an oligochitosan or a derivative thereof having a molecular weight of at least 1 KDa and a degree of deacetylation of at least 1%. The methods described herein have numerus applications with respect to the treatment or prevention of viral infections as well as the detection and quantification of viral load. Also described herein are the synthesis of the oligochitosan and derivatives thereof.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-D illustrate schematics of chemical modifications of NCD1. Synthetic pathways followed to prepare NCD5 (1A), NCD7 (1B) and NCD11 (1C) analogs as described in the methods section. Synthetic pathway followed to prepare each of the analogs as described in the methods section is provided (1D).

FIG. 2 shows the chemical structures of representative derivatives described herein.

FIGS. 3A-3B show NCD1 neutralization of RSV and Coxsackievirus. A culture supernatant was spiked with either RSV or coxsackievirus (FIG. 3A). RNA was isolated from supernatant after incubation. The RNA from supernatant containing virus and treated with PBS served as control. The total RNA was isolated from the pellets representing complexes of NCD1 and RSV, as shown in FIG. 3B. The results show that the viral RNA was recovered from the pellet. The assay was done in triplicate. The results of a representative experiment of N=3 are shown. *, P=<0.05 and **, P=<0.01

FIGS. 4A-4B show inactivation of SARS-COV-2 following NCD1-TPP binding to the virus. NCD1-TPP was used to absorb the virus (recombinant SARS-COV-2 expressing green fluorescence reporter protein) from the culture supernatant. Thus, NCD1-TPP was incubated with SARS-COV-2 for 30 min and added onto Calu3 lung cells plated in 6 well plate (300,000 cells/well). The equal amount of virus incubated in PBS was used as control to infect Calu3 cells. Forty-eight hours post infection the viral supernatant was collected and used to reinfect a fresh batch of Calu3 cells. and treated similarly and the pellet added onto Calu3 cells. After a further 48 hrs post infection, cells were examined for infection by fluorescent microscopy (FIG. 4A) and the RNA was examined for SARS-CoV2 Spike and N gene expression by qPCR (FIG. 4B).

FIG. 5 shows the effect of NCD1-treatment in HIV-infected PBMCs. PBMCs were treated with NCD1 or not treated, followed by infection with HIV-1 89.6. Samples were pelleted on days 0, 2, and 3. Culture supernatants were assayed for p24 concentration. The assay was done in triplicate. The results of a representative experiment of N=3 are shown. *, P=<0.05 and **, P=<0.01.

FIG. 6 shows a screening of compounds for HIV binding in culture supernatant, as determined by total RNA, which may be used to select such compounds for use in detection or therapeutic treatment of HIV infections. The desired compound in PBS or DMSO was incubated with virus for 30 min and then centrifuged. Total RNA isolated from supernatant was quantified. The assay was done in triplicate. The results of a representative experiment of N=3 are shown. *, P=<0.05 and **, P=<0.01

FIG. 7 shows screening of compounds for HIV binding in culture supernatant, as determined by p24 assay, which may be used to select such compounds for use in detection or therapeutic treatment of HIV infections. The assay was done in triplicate. The results of a representative experiment of N=3 are shown. *, P=<0.05 and **, P=<0.01

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “an excipient” include, but are not limited to, mixtures or combinations of two or more such excipients, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “R¹,” “R²,” “X,” and “Y” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “alkanediyl” as used herein, refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, and —CH₂CH₂CH₂— are non-limiting examples of alkanediyl groups.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹—(OA²)ₐ—OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “ Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl. Fused aryl groups including, but not limited to, indene and naphthalene groups are also contemplated.

The term “aldehyde” as used herein is represented by the formula —C(O)H. Throughout this specification “C(O)” is a short hand notation fora carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. An unsubstituted amino group has the structure is —NH₂. A substituted amino group has the structure —NA¹A², where A¹ and/or A² are not hydrogen.

The term “alkylamino” as used herein is represented by the formula —NH(-alkyl) and —N(-alkyl)₂, where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen” or “halide,” as used herein can be used interchangeably and refer to F, CI, Br, or I.

The terms “pseudohalide,” “pseudohalogen” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxy” as used herein is represented by the formula —OH.

The term “alkylhydroxy” as used herein is an alkyl group with one or more hydrogen atoms substituted with a hydroxy group.

The term “arylalkoxy group” as used herein is an alkoxy group with one or more hydrogen atoms substituted with an aryl group.

The term “alkylcarboxylic acid” as used herein is an alkyl group with one or more hydrogen atoms substituted with a carboxylic acid group.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A₂, where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

The term “alkylthiol” as used herein is an alkyl group with one or more hydrogen atoms substituted with a thiol group.

“R¹,” “R²,” “R³,” . . . “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

It is also appreciated that certain compounds described herein can be present as an equilibrium of tautomers. For example, ketones with an a-hydrogen can exist in an equilibrium of the keto form and the enol form.

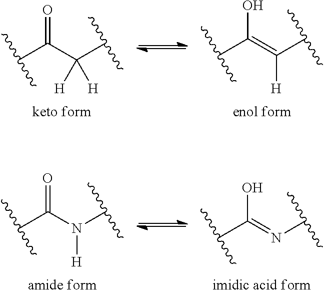 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the invention includes all such possible tautomers.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. One or more therapeutic agents can be administered concurrently or sequentially with the oligochitosan and derivatives thereof described herein. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, “vertebrate subject” is any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered.

A subject in need of “reduced viral load” is one that has been identified as being at risk for developing disease or having developed disease.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, the term “effective amount” refers to an amount that is sufficient to achieve the desired modification of a physical property of the composition or material. For example, an “effective amount” of an oligochitosan or derivative thereof refers to an amount that is sufficient to achieve the desired improvement in the property modulated by the formulation component, e.g. reducing viral load. The specific level in terms of wt % in a composition required as an effective amount will depend upon a variety of factors including the amount and type of compound, type of cell or tissue, co-administration of additional therapies, and type of cancer or other disorder that is to be treated.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non- toxic esters of the present disclosure include C 1 -to-C 6 alkyl esters and C 5 -to-C 7 cycloalkyl esters, although C 1 -to-C 4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C 1 -to-C 6 alkyl amines and secondary C 1 -to-C 6 dialkyl amines. In the case of secondary amines, the amine can also be in the form of a 5- or 6-membered heterocycle containing one nitrogen atom. Amides derived from ammonia, C 1 -to-C 3 alkyl primary amides and C 1 -to-C 2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

The term “derivative” is intended to include any suitable modification of the parent molecule of interest or of an analog thereof such as, for example, thiolation, acetylation, glycosylation, phosphorylation, polymer conjugation (such as with polyethylene glycol), or other addition of foreign moieties, so long as the desired biological activity of the parent molecule is retained to at least a significant degree (e.g., such that at least 50% of the desired biological activity of the parent molecule is retained).

The term “fragment” is intended to include a molecule consisting of only a part of the intact full-length sequence and structure. For example, a fragment of a polysaccharide may be generated by degradation (e.g., hydrolysis) of a larger polysaccharide. In one aspect, active fragments of a polysaccharide can include at least about 2-20 saccharide units of the full-length polysaccharide, provided that the fragment in question retains biological activity, such as anticoagulant activity.

The term “purified” generally refers to isolation of a substance such that the substance comprises the majority weight percent of the overall sample. For example, a purified component comprises greater than 50% by weight, preferably 80%-85%, and even more preferably 90-95% of the sample. Techniques for purifying polysaccharides are well-known in the art and include, for example, ion exchange chromatography, affinity chromatography and sedimentation according to density.

The term “contacting” as used herein refers to bringing a disclosed compound or pharmaceutical composition in proximity to a cell, a target protein, or other biological entity together in such a manner that the disclosed compound or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent.

As used herein, a “human immunodeficiency virus” (HIV) are two species of the genus Lentivirus. Human immunodeficiency virus (HIV) types 1 and 2 (HIV-1 and HIV-2) have been identified as the primary etiologic agents of AIDS and its associated disorders.

As used herein, a “paramyxovirus” also referred to as Paramyxoviridae is a family of negative-strand RNA viruses in the order Mononegavirales. An example of a paramyxovirus is respiratory syncytial virus (RSV), also called human respiratory syncytial virus (hRSV) and human orthopneumovirus, which is a very common, contagious virus that causes infections of the respiratory tract.

As used herein, an “orthomyxovirus” also referred to as Orthomyxoviridae is a family of negative-sense RNA viruses. It includes seven genera: Alphainfluenzavirus, Betainfluenzavirus, Deltainfluenzavirus, Gammainfluenzavirus, lsavirus, Thogotovirus, and Quaranjavirus. The first four genera contain viruses that cause influenza in birds and mammals, including humans.

As used herein, a “coronavirus” is a group of related RNA viruses that cause diseases in mammals and birds. In humans and birds, they cause respiratory tract infections that can range from mild to lethal. Mild illnesses in humans include some cases of the common cold (which is also caused by other viruses, predominantly rhinoviruses), while more lethal varieties can cause SARS, MERS, and COVID-19. Coronaviruses constitute the subfamily Orthocoronavirinae, in the family Coronaviridae, order Nidovirales, and realm Riboviria. They are enveloped viruses with a positive-sense single-stranded RNA genome and a nucleocapsid of helical symmetry.

As used herein, a “filovirus” also referred to as Filoviridae is a member of the order Mononegavirales, is the taxonomic home of several related viruses (filoviruses or filovirids) that form filamentous infectious viral particles (virions) and encode their genome in the form of single-stranded negative-sense RNA. Two members of the family that are commonly known are Ebola virus and Marburg virus.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### Methods for Neutralizing Viral Agents

Described herein are methods for neutralizing a virus. The methods involve interacting the virus with an oligochitosan or a derivative thereof having a molecular weight of at least 1 KDa and a degree of deacetylation of at least 1%. Not wishing to be bound by theory, the oligochitosans and derivatives thereof described herein can bind to the viral agent, which is referred to herein as “neutralizing” the viral agent. In one aspect, upon binding with the oligochitosans and derivatives thereof, replication of the virus can be reduced significantly.

The use of the oligochitosans and derivatives thereof described herein have numerus applications with respect to the treatment or prevention of viral infections (i.e., where a virus has been introduced into a subject). In one aspect, the oligochitosans and derivatives thereof can inactivate a virus in a subject, which in turn reduces or prevents the ability the virus to infect the subject. In one aspect, the oligochitosan or the derivative thereof reduces the level or amount of the virus (i.e., viral load) in a bodily fluid of the subject when compared to the same subject not administered the oligochitosan or the derivative thereof. In one aspect, the amount of virus that is reduced in the subject upon administration of the oligochitosan or the derivative thereof is from about 50% to 100%, or about 50%, 55%, 60%, 65%, 70%, 75%, 80%, 85%, 90%, 95%, 99%, or 100%, where any value can be a lower and upper endpoint of a range (e.g., 80% to 95%). In another aspect, the oligochitosan or the derivative thereof can reduce the number of copies of the virus produced when compared to the same subject not administered the oligochitosan or the derivative thereof. The level of detection of the viral load or number of virus copies produced can be determined using techniques known in the art, some of which are provided in the Examples. The level of detection of the viral load or number of virus copies produced can be determined using a number of different types of subject samples such as, for example, blood, serum, saliva, urine, and the like.

In one aspect, disclosed herein is a method for the treatment or prevention of a viral infection in a subject, the method including the step of administering to the subject a therapeutically effective amount of at least one oligochitosan or the derivative thereof, or a pharmaceutically acceptable salt or ester of the oligochitosan or the derivative thereof, or the disclosed pharmaceutical compositions. In some aspects, the subject is a human. In another aspect, the subject has been diagnosed with a need for treatment of the viral infection prior to the administering step. In some aspects, the method further includes the step of identifying a subject in need of treatment of the viral infection.

In another aspect, disclosed herein is a method for reducing the level or amount of the virus in a subject, including the step of administering to the subject a therapeutically effective amount of at least one oligochitosan or the derivative thereof, or a pharmaceutically acceptable salt or ester of the oligochitosan or the derivative thereof, or a disclosed pharmaceutical composition.

In another aspect, disclosed herein is a method for reducing the number of copies of a virus produced in a subject, including the step of administering to the subject a therapeutically effective amount of at least one oligochitosan or the derivative thereof, or a pharmaceutically acceptable salt or ester of the oligochitosan or the derivative thereof, or a disclosed pharmaceutical composition.

The methods described herein have broad applications with respect to the types of viruses that can be neutralized by the oligochitosan or the derivative thereof. In one aspect, the virus is a RNA virus. In another aspect, the virus is a retrovirus. In another aspect, the virus is a human immunodeficiency virus, a paramyxovirus, an orthomyxovirus, a coronavirus, or a filovirus. In one aspect, the oligochitosan or the derivative thereof is useful in treating or preventing conditions including AIDS and other infections. In another aspect, any virus that circulates in the blood, such as influenza, Dengue fever, sexually transmitted diseases and the like, may be treated using the methods described herein. In one aspect, the oligochitosan or the derivative thereof is administered to patients undergoing general treatment with anti-retroviral therapy as an adjuvant therapy. In another aspect, the oligochitosan or the derivative thereof is administered concurrently or sequentially with other drugs such as, for example, antibiotics or antivirals, as an adjuvant therapy. In one aspect, the oligochitosan or the derivative thereof is compounded with another therapeutic such as, for example, an oral antibiotic.

In another aspect, the oligochitosan or a derivative thereof can treat or prevent COVID-19 disease and symptoms thereof. The oligochitosan or a derivative thereof can be administered to a subject in need of reducing viral load in the nose, mouth or in blood.

The amount of the oligochitosan or the derivative thereof administered to the subject can vary. In one aspect, a daily dosage ranging from about 10 mg of the oligochitosan or the derivative thereof for each 1 kg of body weight to about 50 mg of the oligochitosan or the derivative thereof for each 1 kg of body weight may be administered therapeutically to treat an infection in the mammal's blood, reducing the viral load in a mammal's blood to less than 50% of the viral load in the mammal's blood within a short period of hours, provided that the polysaccharide nanoparticles remain in the blood and are not metabolized prior to binding to viruses. The rate of metabolization can be readily determined from testing for comparison between hosts to adjust the therapeutic does with in the operative range.

In one aspect, a therapeutic treatment may be prescribed after detection of a viral agent in a bodily fluid, such as the blood. In one aspect, a patient exhibiting symptoms of AIDS is orally administered a dose of 10 mg/kg to 50 mg/kg of liquid and the post-administration level of HIV in the patient's blood is reduced by up to 99.9% percent after 30 minutes to 240 minutes. In another aspect, treatment continues until less than 200 copies of a virus is detected per milliliter of blood in the subject.

In one aspect, a blood-contacting medical device is coated with one or more of the oligochitosans or the derivatives thereof. In this aspect, the blood is filtered by passing the blood through or across a coated surface within the medical device, which is coated the oligochitosan or the derivative thereof for concentrating viruses on the coated surface within the medical device. In one aspect, the medical device is a disposable membrane, filter or surface. For example, a plurality of membranes may be used that are coated with a layer of the oligochitosan or the derivative thereof bound to the surface of the membranes. The blood of a patient is then directed through the medical device and comes into contact with the membranes, which allow the oligochitosan or the derivative thereof to capture (i.e., neutralize) the virus, cells, or proteins in the blood. The virus, cells, or proteins may be concentrated on the surface of the membrane. In one example, the membrane provides an environment forcing programmed cell death, and the blood is reintroduced into the patient after a delay to induce an immune response.

In another aspect, the oligochitosans or the derivatives thereof are used to concentrate pathogens, such as viruses, within a bodily fluid, for removal or neutralization. By concentrating the virus in the bodily fluid, detection of the virus is greatly enhanced when compared to known methods without resorting to PCR techniques. Non-limiting methods for detecting the virus using the methods described herein are provided in the Examples.

### Oligochitosans and Derivatives Thereof

The oligochitosans and derivatives thereof are derived from chitosan. Chitosan is a linear polysaccharide composed of randomly distributed β-(1→4)-linked D-glucosamine (deacetylated unit) and N-acetyl-D-glucosamine (acetylated unit). It is made by treating the chitin shells of shrimp and other crustaceans with an alkaline substance, such as sodium hydroxide.

The oligochitosan useful in the methods described herein can be synthesized from high molecular weight chitin or chitosan by subjecting chitin or chitosan to an acid, base, heat, or enzymes followed by size exclusion chromatography to produce oligochitosans of varying molecular weight. In one aspect, the oligochitosan has a molecular weight of at least 5 KDa. In another aspect, oligochitosan has a molecular weight of about 5 KDa to about 100 KDa, or about 1 KDa, 5 KDa, 10 KDa, 15 KDa, 20 KDa, 25 KDa, 30 KDa, 35 KDa, 40 KDa, 45 KDa, 50 KDa, 55 KDa, 60 KDa, 65 KDa, 70 KDa, 75 KDa, 80 KDa, 85 KDa, 90 KDa, 95 KDa, or 100 KDa, where any value can be a lower and upper endpoint of a range (e.g., 5 KDa to 15 KDa). The molecular weight of the oligochitosan may be expressed as either a number average molecular weight or a weight average molecular weight and can be measured using gel permeation chromatography or other liquid chromatography techniques. The degree of deacetylation of the oligochitosan can vary as well.

In one aspect, the oligochitosan has a degree of deacetylation of at least 1%, or about 1%, 5%, 10%, 15%, 20%, 25%, 30%, 35%, 40%, 45%, 50%, 55%, 60%, 65%, 70%, 75%, 80%, 85%, 90%, 95%, or 100%, where any value can be a lower and upper endpoint of a range (e.g., 10% to 20%). In one aspect, the oligochitosan having a molecular weight of from about 5 KDa to about 15 KDa and a degree of deacetylation of from about 10% to about 20%. In one aspect, the oligochitosan is essentially free of other chitosan, which essentially allows for levels of impurities that do not substantially affect the properties of the oligochitosan. Non-limiting procedures for producing the oligochitosans described herein are provided in the Examples.

The oligochitosans produced and used herein can be chemically modified to introduce new chemical moieties into the oligochitosan. For example, the oligochitosan can be chemically modified by thiolation, esterification, and methylation. In one aspect, chemical modification of the amino (i.e., deacetylated group) can be performed. In one aspect, the derivative of the oligochitosan includes one or more glucosamine units having the structure I

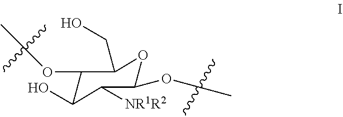

wherein R¹ and R² are independently an alkyl group, an aryl group, a carboxyl group having the formula —C(O)X, where X is an alkyl group, an aryl group, or an alkoxy group, wherein both R¹ and R² are not hydrogen.

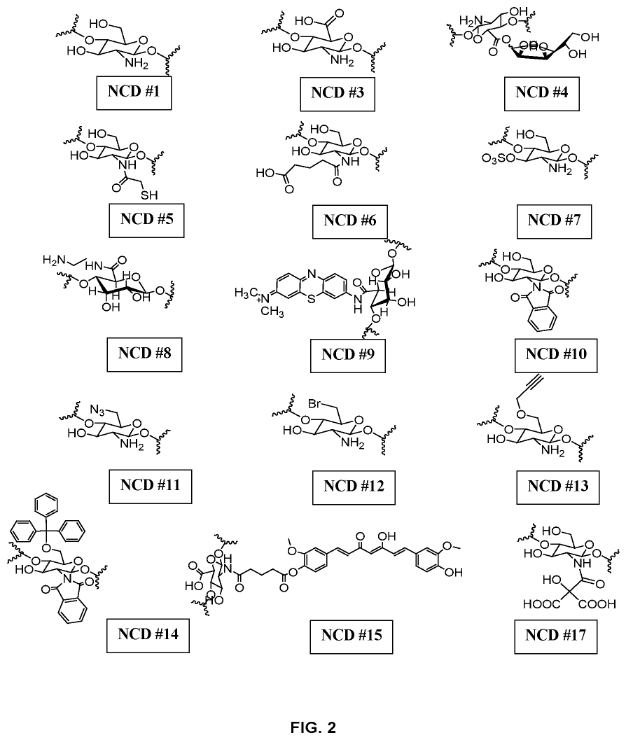

In one aspect, R¹ is hydrogen and R² is —C(O)X, where X is an alkylthiol group. In another aspect, R¹ is hydrogen and R² is —C(O)X, where X is —(CH²)ₙSH, where n is an integer from 1 to 10. A representative compound is NCD5 as provided in FIG. 2.

In another aspect, R¹ is hydrogen and R² is —C(O)X, where X is an alkylcarboxylic acid group or an ester or salt thereof. In one aspect, R¹ is hydrogen and R² is —C(O)X, where X is —(CH₂)ₒCO₂H or the ester or salt thereof, where o is an integer from 1 to 10. In one aspect, R¹ is hydrogen and R² is —C(O)X, where X is an alkyl hydroxy group with one or more carboxylic acid groups. In one aspect, R¹ is hydrogen and R² is —C(O)X, where X is an alkyl hydroxy group with two carboxylic acid groups. Representative compounds are NCD6 and NCD17 as provided in FIG. 2.

Depending upon the reaction conditions, the number of glucosamine units having the structure I can vary and be adjusted accordingly. In one aspect, the derivative of the oligochitosan has from about 10% to about 100% glucosamine units having the structure I, or about 10%, 15%, 20%, 25%, 30%, 35%, 40%, 45%, 50%, 55%, 60%, 65%, 70%, 75%, 80%, 85%, 90%, 95%, or 100%, where any value can be a lower and upper endpoint of a range (e.g., 10% to 20%).

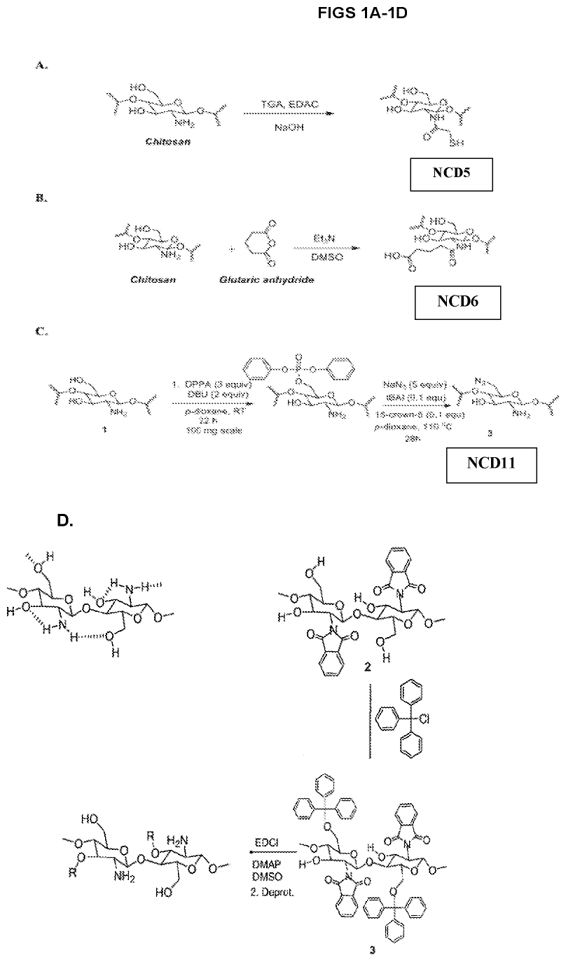

In one aspect, modification of the oligochitosan includes selective protection of an amine group to improve solubility in organic solvents, followed by selective protection of the primary alcohol with a protecting group and subsequent coupling with a desired substituent under standard coupling reaction conditions followed by amine and primary alcohol deprotection. An example of this synthetic route is illustrated in FIG. 1D.

In one aspect, the derivative of the oligochitosan includes one or more glucosamine units having the structure II

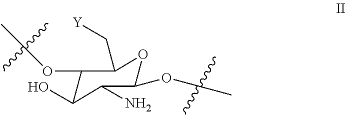

wherein Y is a halo group, an azide group, an alkyl group, an alkenyl group, an alkynyl group, or a carboxyl group having the formula —C(O)X, where X is an alkyl group, an aryl group, an arylalkoxy group, a hydroxyl group, an alkoxy group, or a unsubstituted or unsubstituted amine group.

In one aspect, Y is a bromide group. In another aspect, Y is an azide group. In another aspect, Y is a propargyl group. In another aspect, Y is an aryloxy group comprising one or more phenyl groups. In another aspect, Y is —C(O)X, where X is a hydroxyl group. In another aspect, Y is —C(O)X, where X is —NH(CH₂)ₚNH₂ and p is an integer from 1 to 10. Representative compounds are NCD3, NCDS, NCD8, NCD12, NCD13, NCD14, and NCD17 as provided in FIG. 2.

Depending upon the reaction conditions, the number of glucosamine units having the structure I can vary and be adjusted accordingly. In one aspect, the derivative of the oligochitosan has from about 10% to about 100% glucosamine units having the structure II, or about 10%, 15%, 20%, 25%, 30%, 35%, 40%, 45%, 50%, 55%, 60%, 65%, 70%, 75%, 80%, 85%, 90%, 95%, or 100%, where any value can be a lower and upper endpoint of a range (e.g., 10% to 20%).

In one aspect, the oligochitosan or the derivative thereof can be crosslinked. In one aspect, the use of a crosslinker can be added to the oligochitosan or the derivative thereof. The crosslinker can be any compound that can form a chemical bond (e.g., covalent, ionic, hydrogen, etc.) between two oligochitosan molecules or derivatives thereof. In one aspect, the crosslinker possesses ionic groups that can interact with one or more groups on the oligochitosan. For example, the crosslinker can possess two or more anionic groups that can interact with protonated amino groups on the oligochitosan. In one aspect, the crosslinker includes two or more anionic groups (or groups that can be converted to anionic groups) including, but not limited to, a carboxylate group, a phosphate group, or a sulfonate group.

In one aspect, crosslinker is a polyphosphate, where the polyphosphate an organic compound with two or more phosphate groups or an inorganic polyphosphate. In one aspect, the inorganic polyphosphate can be composed only of phosphate groups. In another aspect, the polyphosphate is composed of 2 to 10 phosphate groups covalently bonded to one another. An example of this is tripolyphosphate (TPP), which has three phosphate groups bonded to one another with a net negative charge of −5. The counterion can be any suitable counterion for pharmaceutical applications. In one aspect, the crosslinker is sodium tripolyphosphate (Na₅P₃O₁₀). In one aspect, the oligochitosan or the derivative thereof can be admixed with the crosslinker to produce the crosslinked product. Non-limiting procedures for producing crosslinked oligochitosan are provided in the Examples.

In one aspect, the oligochitosan or the derivative thereof is not sulfated. In this aspect, none of the hydroxyl groups or the amino groups of the deacetylated are sulfated (i.e., possess an —SO₃ group). Polysaccharides may be chemically altered, for example, to improve anticoagulant function. Such modifications may include sulfation of the polymer. However, the conventional process of preparing sulfated chitosan is disadvantageous in an economical point of view since it requires 5-10 times as much as the theoretical amount of sulfonating agent and a large amount of solvent in the sulfonating reaction step. Additionally, a neutralization step with alkali and addition of a large amount of alcohol to precipitate the dissolving sulfonated chitosan after the reaction step is also required. Furthermore, since the sodium sulfate is also precipitated in a large amount along with the sulfated polymer in the neutralization step, filtration is extremely troublesome and a further separation step such as dialysis is necessary in order to separate the sulfated polymer from the sodium sulfate. This process is not scalable for commercialization. Thus, the preparation of sulfated chitosan remains complex and heterogeneous products make it difficult to use for biological activity.

It has been previously reported that sulfated oligochitosan failed to bind to HIV. [Artan M et al. Carbohyd Res 345:2010: 656-662]. It has been unexpectedly discovered that non-sulfated and unmodified oligochitosan produced herein (e.g., NCD1) exhibited surprising and unexpected binding to HIV.

In one aspect, the oligochitosans and derivatives thereof can be formulated as nanoparticles, which are also referred to herein as “nanochitosans.” Nanonization offers a solution to improve the bioavailability of the poorly soluble drugs. Methods such as milling, high pressure homogenization, vacuum deposition, and high temperature evaporation can be used to produce nanoparticles herein. In other aspects, supercritical fluid-processing (SCF) techniques offer advantages ranging from superior particle size control to clean processing. For example, the use of supercritical CO2 based technologies can produce small particles. Particles that have the smooth surfaces, small particle size and distribution and free flowing can be obtained with particular SCF techniques. Rapid Expansion of Supercritical Solutions (RESS), Supercritical Anti Solvent (SAS) and Particles from Gas Saturated Solutions (PGSS) are three groups of processes which lead to the production of fine and monodisperse powders. RESS involves dissolving a drug in a supercritical fluid (SCF) and passing it through an appropriate nozzle. The SAS processes are based on decreasing the solvent power of a polar organic solvent in which the substrate (API & polymer of interest) is dissolved, by saturating it with carbon dioxide (CO2) at supercritical conditions. CO2 causes precipitation and recrystalization of the drug. SAS is scalable and can be applied to a wide variety of APIs and polymers. In PGSS, CO2 is dissolved in organic solutions or melted compounds and it is successfully used for manufacturing drug products as well as for drying purposes. In one aspect, the nanoparticles have a diameter of from about 1 nm to about 100 nm, or about 1 nm, 5 nm, 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, or 100 nm, where any value can be a lower and upper endpoint of a range (e.g., 30 nm to 80 nm).

In one aspect, the oligochitosans and derivatives thereof can be isolated as solvates and, in particular, as hydrates of a disclosed compound, which can be obtained, for example, by crystallization from a solvent or from aqueous solution. In this connection, one, two, three or any arbitrary number of solvate or water molecules can combine with the compounds according to the invention to form solvates and hydrates.

The oligochitosans and derivatives thereof can be used in the form of salts derived from inorganic or organic acids. Pharmaceutically acceptable salts include salts of acidic or basic groups present in the disclosed compounds. Suitable pharmaceutically acceptable salts include base addition salts, including alkali metal salts, e.g., sodium or potassium salts; alkaline earth metal salts, e.g., calcium or magnesium salts; and salts formed with suitable organic ligands, e.g., quaternary ammonium salts, which may be similarly prepared by reacting the drug compound with a suitable pharmaceutically acceptable base. The salts can be prepared in situ during the final isolation and purification of the compounds of the present disclosure; or following final isolation by reacting a free base function, such as a secondary or tertiary amine, of a disclosed compound with a suitable inorganic or organic acid; or reacting a free acid function, such as a carboxylic acid, of a disclosed compound with a suitable inorganic or organic base.

Acidic addition salts can be prepared in situ during the final isolation and purification of a disclosed compound, or separately by reacting moieties comprising one or more nitrogen groups with a suitable acid. In various aspects, acids which may be employed to form pharmaceutically acceptable acid addition salts include such inorganic acids as hydrochloric acid, sulfuric acid and phosphoric acid and such organic acids as oxalic acid, maleic acid, succinic acid and citric acid. In a further aspect, salts further include, but are not limited, to the following: hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, butyrate, camphorate, camphorsulfonate, digluconate, glycerophosphate, hemisulfate, heptanoate, hexanoate, fumarate, hydrochloride, 2-hydroxyethanesulfonate (isethionate), nicotinate, 2-naphthalenesulfonate, oxalate, pectinate, persulfate, 3-phenylpropionate, picrate, pivalate, propionate, succinate, tartrate, thiocyanate, phosphate, glutamate, bicarbonate, undecanoate, and pamoate (i.e., 1,1′-methylene-bis-(2-hydroxy-3-naphthoate)) salts. Also, basic nitrogen-containing groups can be quatemized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others.

Basic addition salts can be prepared in situ during the final isolation and purification of a disclosed compound, or separately by reacting carboxylic acid moieties with a suitable base such as the hydroxide, carbonate or bicarbonate of a pharmaceutical acceptable metal cation or with ammonia, or an organic primary, secondary or tertiary amine. Pharmaceutical acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like. In further aspects, bases which may be used in the preparation of pharmaceutically acceptable salts include the following: ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

### Pharmaceutical Compositions

In various aspects, the present disclosure relates to pharmaceutical compositions comprising a therapeutically effective amount of at least one oligochitosan or derivative thereof, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one oligochitosan or derivative thereof or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitoneally, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

Pharmaceutically acceptable salts can be prepared from pharmaceutically acceptable non-toxic bases or acids. For therapeutic use, salts of the disclosed compounds are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms which the disclosed compounds are able to form.

In various aspects, an oligochitosan or derivative thereof comprising an acidic group or moiety, e.g., a carboxylic acid group, can be used to prepare a pharmaceutically acceptable salt. For example, such a disclosed compound may comprise an isolation step comprising treatment with a suitable inorganic or organic base. In some cases, it may be desirable in practice to initially isolate a compound from the reaction mixture as a pharmaceutically unacceptable salt and then simply convert the latter back to the free acid compound by treatment with an acidic reagent, and subsequently convert the free acid to a pharmaceutically acceptable base addition salt. These base addition salts can be readily prepared using conventional techniques, e.g., by treating the corresponding acidic compounds with an aqueous solution containing the desired pharmacologically acceptable cations and then evaporating the resulting solution to dryness, preferably under reduced pressure. Alternatively, they also can be prepared by mixing lower alkanolic solutions of the acidic compounds and the desired alkali metal alkoxide together, and then evaporating the resulting solution to dryness in the same manner as before.

Bases which can be used to prepare the pharmaceutically acceptable base-addition salts of the base compounds are those which can form non-toxic base-addition salts, i.e., salts containing pharmacologically acceptable cations such as, alkali metal cations (e.g., lithium, potassium and sodium), alkaline earth metal cations (e.g., calcium and magnesium), ammonium or other water-soluble amine addition salts such as N-methylglucamine-(meglumine), lower alkanolammonium and other such bases of organic amines. In a further aspect, derived from pharmaceutically acceptable organic non-toxic bases include primary, secondary, and tertiary amines, as well as cyclic amines and substituted amines such as naturally occurring and synthesized substituted amines. In various aspects, such pharmaceutically acceptable organic non-toxic bases include, but are not limited to, ammonia, methylamine, ethylamine, propylamine, isopropylamine, any of the four butylamine isomers, betaine, caffeine, choline, dimethylamine, diethylamine, diethanolamine, dipropylamine, diisopropylamine, di-n-butylamine, N,N′-dibenzylethylenediamine, pyrrolidine, piperidine, morpholine, trimethylamine, triethylamine, tripropylamine, tromethamine, 2-diethylaminoethanol, 2-dimethylaminoethanol, ethanolamine, quinuclidine, pyridine, quinoline and isoquinoline; benzathine, N-methyl-D-glucamine, ethylenediamine, N-ethylmorpholine, N-ethylpiperidine, glucamine, glucosamine, methylglucamine, morpholine, piperazine, piperidine, polyamine resins, procaine, purines, theobromine, hydrabamine salts, and salts with amino acids such as, for example, histidine, arginine, lysine and the like. The foregoing salt forms can be converted by treatment with acid back into the free acid form.

In various aspects, an oligochitosan or derivative thereof comprising a protonatable group or moiety, e.g., an amino group, can be used to prepare a pharmaceutically acceptable salt. For example, such a disclosed compound may comprise an isolation step comprising treatment with a suitable inorganic or organic acid. In some cases, it may be desirable in practice to initially isolate a compound from the reaction mixture as a pharmaceutically unacceptable salt and then simply convert the latter back to the free base compound by treatment with a basic reagent, and subsequently convert the free base to a pharmaceutically acceptable acid addition salt. These acid addition salts can be readily prepared using conventional techniques, e.g., by treating the corresponding basic compounds with an aqueous solution containing the desired pharmacologically acceptable anions and then evaporating the resulting solution to dryness, preferably under reduced pressure. Alternatively, they also can be prepared by treating the free base form of the disclosed compound with a suitable pharmaceutically acceptable non-toxic inorganic or organic acid.

Acids that can be used to prepare the pharmaceutically acceptable acid-addition salts of the base compounds are those which can form non-toxic acid-addition salts, i.e., salts containing pharmacologically acceptable anions formed from their corresponding inorganic and organic acids. Exemplary, but non-limiting, inorganic acids include hydrochloric hydrobromic, sulfuric, nitric, phosphoric and the like. Exemplary, but non-limiting, organic acids include acetic, benzenesulfonic, benzoic, camphorsulfonic, citric, ethanesulfonic, fumaric, gluconic, glutamic, isethionic, lactic, maleic, malic, mandelicmethanesulfonic, mucic, pamoic, pantothenic, succinic, tartaric, p-toluenesulfonic acid and the like. In a further aspect, the acid-addition salt comprises an anion formed from hydrobromic, hydrochloric, maleic, phosphoric, sulfuric, and tartaric acids.

In practice, the oligochitosans and derivatives thereof or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise an oligochitosan or derivative thereof (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The oligochitosans or derivatives thereof described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42 p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik and angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions or suspensions, including saline and buffered media. Parenteral vehicles can include mannitol, normal serum albumin, sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present invention may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present invention include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the invention formulated in a pharmaceutically acceptable carrier may also be prepared, placed in an appropriate container, and labeled for treatment of an indicated condition.

Another patch system configuration which can be used by the present invention is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present invention is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The disclosed pharmaceutical compositions can further comprise other therapeutically active compounds, which are usually applied in the treatment of the above mentioned pathological or clinical conditions.

### Kits

In a further aspect, the present disclosure relates to kits comprising at least one oligochitosan or derivative thereof and instructions for administering the oligochitosan or derivative thereof in connection with treating or preventing a viral infection.

The oligochitosan or derivative thereof and/or pharmaceutical compositions comprising the same can conveniently be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient. In further aspects, a kit can include optional components that aid in the administration of the unit dose to patients, such as vials for reconstituting powder forms, syringes for injection, customized IV delivery systems, inhalers, etc. Additionally, a kit can contain instructions for preparation and administration of the compositions. The kit can be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

In a further aspect, the disclosed kits can be packaged in a daily dosing regimen (e.g., packaged on cards, packaged with dosing cards, packaged on blisters or blow-molded plastics, etc.). Such packaging promotes products and increases patient compliance with drug regimens. Such packaging can also reduce patient confusion. The present invention also features such kits further containing instructions for use.

In a further aspect, the present disclosure also provides a pharmaceutical pack or kit comprising one or more containers filled with one or more of the ingredients of the pharmaceutical compositions described herein. Associated with such container(s) can be a notice in the form prescribed by a governmental agency regulating the manufacture, use or sale of pharmaceuticals or biological products, which notice reflects approval by the agency of manufacture, use or sale for human administration.

In various aspects, the disclosed kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

### Aspects

Aspect 1. A method for treating or preventing a viral infection in a subject comprising administering to the subject an oligochitosan or a derivative thereof or a pharmaceutically acceptable salt or ester thereof having a molecular weight of at least 1 KDa and a degree of deacetylation of at least 1%.

Aspect 2. The method of Aspect 1, wherein the oligochitosan or a derivative thereof has a molecular weight of from about 1 KDa to about 100 KDa.

Aspect 3. The method of Aspects 1 or 2, wherein the oligochitosan or a derivative thereof has a degree of deacetylation of from about 10% to 100%.

Aspect 4. The method in any one of Aspects 1 to 3, wherein the subject is administered oligochitosan having a molecular weight of from about 5 KDa to about 15 KDa and a degree of deacetylation of from about 10% to about 20%.

Aspect 5. The method in any one of Aspects 1 to 4, wherein the derivative of the oligochitosan comprises one or more glucosamine units having the structure I

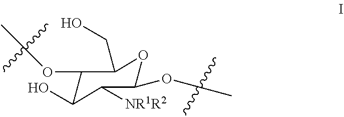

wherein R¹ and R² are independently an alkyl group, an aryl group, a carboxyl group having the formula —C(O)X, where X is an alkyl group, an aryl group, or an alkoxy group, wherein both R¹ and R² are not hydrogen.

Aspect 6. The method of Aspect 5, wherein R¹ is hydrogen and R² is —C(O)X, where X is an alkylthiol group.

Aspect 7. The method of Aspect 5, wherein R¹ is hydrogen and R² is —C(O)X, where X is —(CH₂)ₙSH, where n is an integer from 1 to 10.

Aspect 8. The method of Aspect 5, wherein R¹ is hydrogen and R² is —C(O)X, where X is an alkylcarboxylic acid group or an ester or salt thereof.

Aspect 9. The method of Aspect 5, wherein R¹ is hydrogen and R² is —C(O)X, where X is —(CH₂)ₒCO₂H or the ester or salt thereof, where o is an integer from 1 to 10.

Aspect 10. The method of Aspect 5, wherein R¹ is hydrogen and R² is —C(O)X, where X is an alkyl hydroxy group with one or more carboxylic acid groups.

Aspect 11. The method of Aspect 5, wherein R¹ is hydrogen and R² is —C(O)X, where X is an alkyl hydroxy group with two carboxylic acid groups.

Aspect 12. The method of Aspect 5, wherein the derivative of the oligochitosan comprises from 10% to 100% glucosamine units having the structure I.

Aspect 13. The method in any one of Aspects 1 to 4, wherein the derivative of the oligochitosan comprises one or more glucosamine units having the structure II

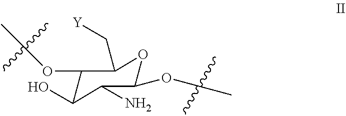

wherein Y is a halo group, an azide group, an alkyl group, an alkenyl group, an alkynyl group, or a carboxyl group having the formula —C(O)X, where X is an alkyl group, an aryl group, an arylalkoxy group, a hydroxyl group, an alkoxy group, or a unsubstituted or unsubstituted amine group.

Aspect 14. The method of Aspect 13, wherein Y is a bromide group.

Aspect 15. The method of Aspect 13, wherein Y is an azide group.

Aspect 16. The method of Aspect 13, wherein Y is a propargyl group.

Aspect 17. The method of Aspect 13, wherein Y is an aryloxy group comprising one or more phenyl groups.

Aspect 18. The method of Aspect 13, wherein Y is —C(O)X, where X is a hydroxyl group.

Aspect 19. The method of Aspect 13, wherein Y is —C(O)X, where X is —NH(CH₂)ₚNH₂ and p is an integer from 1 to 10.

Aspect 20. The method of Aspect 13, wherein the derivative of the oligochitosan comprises from 10% to 100% glucosamine units having the structure II.

Aspect 21. The method in any one of Aspects 1 to 20, wherein the oligochitosan or the derivative thereof is crosslinked.

Aspect 22. The method of Aspect 21, wherein the oligochitosan or the derivative thereof is crosslinked with a polyphosphate.

Aspect 23. The method of Aspect 21, wherein the oligochitosan or the derivative thereof is crosslinked with a tripolyphosphate.

Aspect 24. The method in any one of Aspects 1 to 23, wherein the oligochitosan or the derivative thereof is not sulfated.

Aspect 25. The method in any one of Aspects 1 to 24, wherein the oligochitosan or the derivative thereof comprises nanoparticles.

Aspect 26. The method in any one of Aspects 1 to 25, wherein the oligochitosan or the derivative thereof is administered as a pharmaceutical composition.

Aspect 27. The method of Aspect 26, wherein the pharmaceutical composition further comprises an antibiotic, an antiviral, or a combination thereof.

Aspect 28. The method in any one of Aspects 1 to 27, wherein the subject is a mammal.

Aspect 29. The method in any one of Aspects 1 to 27, wherein the subject is a human.

Aspect 30. The method in any one of Aspects 1 to 27, wherein the subject has been diagnosed with a need for treatment of the viral infection prior to the administering step.

Aspect 31. The method in any one of Aspects 1 to 27, further comprising the step of identifying a subject in need of treatment of the viral infection.

Aspect 32. The method in any one of Aspects 1 to 31, wherein the viral infection is caused by a RNA virus.

Aspect 33. The method in any one of Aspects 1 to 31, wherein the viral infection is caused by a retrovirus.

Aspect 34. The method in any one of Aspects 1 to 31, wherein the viral infection is caused by SARS-CoV2.

Aspect 35. The method in any one of Aspects 1 to 31, wherein the viral infection is caused by a human immunodeficiency virus, a paramyxovirus, an orthomyxovirus, a coronavirus, or a filovirus.

Aspect 36. The method in any one of Aspects 1 to 35, wherein the subject is further administered an antibiotic, an antiviral, or a combination thereof.

Aspect 37. The method in any one of Aspects 1 to 36, wherein the oligochitosan or the derivative thereof reduces the level or amount of the virus in the subject.

Aspect 38. The method in any one of Aspects 1 to 37, wherein the oligochitosan or the derivative thereof neutralizes the virus in the subject.

Aspect 39. A medical device comprising a coating of an oligochitosan or a derivative thereof having a molecular weight of at least 1 KDa and a degree of deacetylation of at least 1%.

Aspect 40. The medical device of Aspect 39, wherein the device comprises a membrane or filter.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure.

Material and Methods

Viral Cultures: HIV89.6 was obtained from NIH NIAID repository.

Compounds used in the study. NCD1, carboxylated NCD1 (NCD3), ester mannosylated NCD1 (NCD4), N-thiolated (NCDS), (N-glutamyl NCD1(NCD6), Sulfated NCD1 (NCD7), Oxidated diamino Starch (NCD8), Starch azure (NCD9), N-Phthaloyl NCD1(NCD10), Azido NCD1(NCD11), Bromo NCD1(NCD12), Propargyl NCD1 (NCD13), N3-phthuloyl NCD1(NCD 14), curcumin-glutaryl NCD1 (NCD 15), Thiolated BPMH NCD1 (NCD 16) and Nitric NCD1 (NCD 17) whose structures are shown in FIG. 2.

Stock solutions of 10 mg/ml of NCD1 were prepared in water. Stock solutions (10 mg/ml) of rest of the compounds were prepared using 10% DMSO in PBS. Respective negative controls were used in the study.

HIV p24 Assay: HIV-1 p24 ELISA was performed using HIV-1 p24 antigen capture assay according to manufacturer's protocol (Advanced BioSciences Laboratory).

Real-time PCR assay for the detection and quantitation of HIV-1 from cell culture.

Medium/blood samples. To ensure uniformity and reproducibility, standards with known viral copy numbers of HIV89.6 (NIH NIAID repository) were prepared. A known amount of virus was spiked into cell culture medium in ten-fold serial dilutions. HIV-1 RNA was extracted using the High Pure viral RNA kit (Ambion). Complementary DNA (cDNA) was synthesized with the SuperScript III cDNA Synthesis Kit (Invitrogen) and amplified and quantified using primers specific for HIV-1 LTR. Real-time PCR amplification was performed in a CFX96™ Real-Time system (Bio-Rad, Hercules, CA). The primers used were specific to a conserved region of HIV-1 LTR: 5′-GRAACCCACTGCTTAASSCTCAA-3′ [SEQ. ID. NO. 1] (LTR sense; position 506 of HxB2); 5′-TGTTCGGGCGCCACTGCTAGAGA-3′ [SEQ. ID. NO. 2] (LTR antisense; position 626 of HxB2). See Mehta, N et. al, PLoS One. 2009 Jun. 5; 4(6):e5819, which is incorporated herein by reference in its entirety. HIV-1 specific amplicons were detected using SYBR Green (Bio-Rad). The number of HIV-1 RNA copies in each test template was measured by its threshold cycle (Ct) as determined from the curve of serially diluted standards using data analysis software (Bio-Rad CFX Manager). The threshold cycle values were plotted against copy numbers to construct the standard curve. Quantification of HIV-1 RNA in each test sample was back calculated and viral load was expressed as copies/ml. At the end of the assay, the specificity of each amplified product was ascertained by means of melting curve analysis. This eliminated false-positive detections due to primer-dimers or nonspecific amplicons.

p24 ELISA and quantitative PCR from human blood. The compounds NCD1, NCD3, NCD4, NCD5, NCD6, NCD7, NCD8, NCD9, NCD10, NCD11, NCD12, NCD13, NCD14, NCD15 and NCD17 were tested for their efficacy in binding to the HIV-89.6 virus in the presence of blood. Human whole blood was acquired from Florida Blood Services and each time 100 μl of blood was spiked with 1 mg of the compound and 35 pg of HIV-89.6. The mix was incubated for 30 min at room temperature on a rotating platform. Later supernatant was separated upon centrifugation (12000 rpm for 5 minutes). Part of the supernatant was used to determine p24 levels using HIV-1 p24 antigen capture assay (Advanced Bioscience Laboratory). Remaining supernatant was used for isolation of RNA using High Pure viral RNA isolation kit (Roche). 5 μl of the isolated RNA was used to synthesize complimentary DNA; HIV-1 LTR was synthesized using Superscript III (Invitrogen).

Quantitative real time PCR was performed using BioRad CFX 96 with the known amounts of HIV-1 RNA as standards. The starting quantity of HIV-1 RNA in the samples treated with different compounds was determined using the standard curve and is plotted on the graph.

Inhibition of Replication of SARS-CoV-2 Virus

To test the NCD1 inhibition of SARS-CoV-2 replication, Calu3 cells were plated (300, 000/well) in a 6 well plate. One day after, Calu3 cells were infected with either 1 MOI SARS CoV2 virus or with 1 MOI SARS CoV2 virus preincubated (for 30) mins with 10 ug/ml TPP-NCD1. Forty-eight hours post infection the viral supernatant was collected and was used to reinfect a fresh batch of Calu3 cells. After a further 48 hrs post infection, cells were examined for infection by fluorescent microscopy and the RNA was examined for SARS-CoV2 Spike and N gene expression by qPCR.

### Example 1. Preparation and Characterization of Oligochitosan (NCD1)

Oligochitosan 10 kDa, a soluble oligochitosan, (OC10) (10 mg/ml) was prepared by acid hydrolysis of insoluble chitosan (purchased from Sigma) and the products of the oligochitosan 10 KDa size were characterized by C13 NMR and FT-IR. The oligomeric chitosan polysaccharide was obtained as yellowish solid was characterized by ¹H, ¹³C NMR and FT-IR. ¹H NMR (400 MHz, D₂O): δ 3.72 (5H, m), 3.59-3.53 (3H, m), 2.97 (2H, t), 1.88 (1H, s). ¹³C NMR (100 MHz, D₂O): δ 177.24, 101.0, 97.77, 76.49, 74.92, 70.27, 60.06, 55.93, 22.24, 20.50-19.72. FTIR: 3340 (O—H stretch); 1620.8 (Allyl C═C); 1514, 1507 and 1378.3 (aromatic C═O, N—H bending & C—N stretching).

### Example 2. Synthesis of Chitosan Derivatives

In general, when the modification of OC10 was performed at a different position other than N, the procedure involved protection and deprotection. The amino group was accomplished using phthalic anhydride in dry dimethylformamide (DMF) for 12 h and reflux. Alternatively, double protection was utilized at the amino position and the C6 primary alcohol position. If not soluble in water, a solubilizer, such as DMSO may be used to dissolve a composition for testing. The synthetic procedures provided below are not exhaustive and not be limited to the specific examples.

Some of the derivatives described herein show good activity for clearing high levels of viral load from serum, particularly NCD5 and NCD6. FIGS. 1A-1D provide non-limiting synthetic procedures for making derivatives described herein. For example, 500 mg of oligochitosan NCD1 was dissolved in 5 ml of DMSO and 0.39 g, 3.43 mmol of glutaric anhydride was added and a catalytic amount of DMAP followed by 740 μl, 5.28 mmol of triethylamine. The reaction was run overnight. The crude product was precipitated in ice-cold water and washed with 3×10 ml with cold water and then resuspended in a mixture ethanol-water and dialyzed ×24 h and finally freeze-dried ×24 h. The final product, a whitish solid, was obtained in a 54% yield and confirmed by NMR and FTIR.

For thiolated-chitosan analog (NCD5), the final product, a white solid was obtained in a 67% yield and confirmed by NMR and FTIR. ¹H NMR and FT-IR. ¹H NMR (400 MHz, D₂O): δ 3.72 (5H, m), 3.59-3.54 (3H, m), 2.99 (2H, t), 1.87(1H, s), 1.10-0.9 (2H, m), 0.52(1H, s). ¹³C NMR (100 MHz, D₂O): δ 177.24, 101.0, 97.77, 76.49, 74.92, 70.27, 60.06, 55.93, 47.90, 33.33, 22.24, 20.50-19.72. FTIR: 3225 (O—H stretch); 2496 (—S—H stretching); 1681 (N—H deformation); 1546 (amide II), 1246 (C—SH stretching), 1149 and 1032 (asymmetric stretching vibration C—O—C), 808 (S—S bisulfide bond).

### Example 3.1 Synthesis of Glutaryl-Chitosan Derivative (NCD6)

To synthesize NCD6 (FIG. 1B), 500 mg of chitosan OC10 was dissolved in 15 ml of water acidulated with 1% acetic acid. Then 500 μl of TGA was added followed by the addition of 0.5 g, 2.6 μmol of EDCI and a catalytic amount of DMAP. The reaction was run overnight. The resulting product was precipitated and washed with 3×5 ml cold ethanol. The product was then resuspended in water and dialyzed for 24 hours changing the water every 4 hr. The final product, a white solid, was obtained in a 67% yield and confirmed by NMR and FTIR.

The final product, a whitish solid, was obtained in a 54% yield and confirmed by NMR and FTIR. ¹H NMR (400 MHz, DMSO-d6): δ 7.95-7.92 (1H, s), 2.89 (1H, s), 2.73 (1H, s), 2.54-2.50 (4H, m), 2.29-2.22 (1H, m), 1.72-1.67 (2H, t). ¹³C NMR (100 MHz, DMSO-d6): δ 182.50, 141.35, 138.41, 134.20, 133.08, 129.26, 128.13, 126.61, 97.66, 89.03, 76.50, 74.96, 70.23, 60.22, 56.03, 22.3611. FTIR: 3340 (O—H stretch); 1705 (C═O stretch); 1642 (N—H deformation); 1546 (amide II), 1378, 1150 and 1000 (asymmetric stretching vibration C—O—C).

### Example 3.2 Synthesis of Azido-Chitosan Analog (NCD11)

A white solid was obtained in a 78% yield and confirmed by ¹³C, ¹H NMR and FTIR studies. ¹H NMR (400 MHz, D₂O): δ 3.72 (5H, m), 3.59-3.54 (3H, m), 2.99 (2H, t), 1.87(1H, s). ¹³C NMR (100 MHz, D₂O): δ 177.24, 101.0, 97.77, 76.49, 74.92, 70.27, 60.06, 55.93, 22.24, 20.50-19.72. FTIR: 3340 (O—H stretch); 2361-2341 (—N═N⁺═N strong vibration); 1681 (N—H deformation); 1546 (amide II), 1379, 1149 and 1032 (asymmetric stretching vibration C—O—C).

### Example 3.3

Additional NCD1 analogs are illustrated in FIG. 2. The compounds synthesized involved carboxylation (NCD3), ester mannosylation (NCD4), N-thiolation (NCDS), N-glutamyl addition (NCD6), sulfation (NCD7), oxidation with diamino starch (NCD8), oxidation with starch azure (NCD9), N-phthaloyl (NCD10), azido-methyl (NCD11), bromination (NCD12), propargylation (NCD13), N-phthaloylation (NCD14), conjugation with curcumin glutaryl (NCD15), and amidation using citric acid (NCD17) whose structures are shown in FIG. 2.

### Example 3.4 NCD11 Synthesis

In general, when the modification was done in a different position other than N, the procedure involved protection and deprotection of the amino group such as NCD11, NCD12, and NCD13, usually using phthalic anhydride in dry dimethylformamide (DMF) for 12 h and reflux. In some other cases, it was necessary to do double protection at the amino position and the C6 primary alcohol. The general scheme of protection and deprotection is shown in FIG. 1D.

For example, an additional procedure was used to prepare an alternative derivative equally useful or even more convenient. To synthesize NCD11 (FIG. 1C), the starting OC10 chitosan (100 mg) was suspended in dry 1,4 p-dioxane (0.5M) at room temperature under argon atmosphere with magnetic stirring. DPPA (6.5 μl, 1.8 equiv) and DBU (3.0 μl, 2.4 equiv) were then added drop wise. The reaction was usually completed overnight. The reaction mixture was then heated under argon atmosphere in an oil bath after the addition of sodium azide (5 equiv) and 15-crown-5 (0.1 equiv). The reaction mixture was allowed to stir overnight for completion. The solvent was evaporated and the yellow solid was suspended in water (3×20 ml) and the solid recovered by centrifugation at 4K g, then washed with EtOAc (3×20 ml) and the solid recovered by centrifugation at 4K g and finally washed with ethanol and acetone and the solid recovered as described before. With this procedure 100 mg of the final product was produced. The product was confirmed by ¹³C and ¹H NMR studies.

The last compound that has presented a noticeable activity corresponds to curcuminyl glutaryl chitosan (NCD16), a synthesis reaction illustrated in FIG. 2D. In a 50 ml round bottom flask 30 mg, 0.06 mmol of glutaryl curcurnin analog was placed and 15 mg, 0.081 mmol of EDCI and a catalytic amount of DMAP and dissolved with 5 ml of dry DMSO. Stir during the reaction for 10 min, then add 120 mg of the OC10, which was pre-dissolved in water (minimum amount necessary). This was allowed to react overnight and its progress monitored by TLC in MeOH:DCM [1:9]. Once the reaction was finished, the crude product was obtained by precipitation in cold ethanol, and was 3×10 ml of the same solvent. The resultant precipitated was redissolved in DI water and dialyzed against water ×24 hours and finally freeze-dried for another 24 hours, providing 87 mg of a yellow solid. The compound was confirmed by FTIR and NMR.

### Example 3.5 Synthesis of TPP-NCD1

A solution of 2 mg/mL NCD1 was dispersed in sterile H₂O. A stock of the Tripolyphosphate Penta-sodium (TPP) (Sigma-Aldrich Co), solution was dissolved in sterile H₂O at a concentration of 10 mg/mL. 1 mL of NCD1 solution was taken in the V-bottom vial and 20 μL of TPP from stock was added to the NCD1 solution, the solution was to ultrasonication for 15 minutes. The NP solution was then centrifuged, and loaded NPs were stored at 4° C. until further use.

### Example 4. NCD1 Enabled Neutralization of RSV and Coxsackievirus

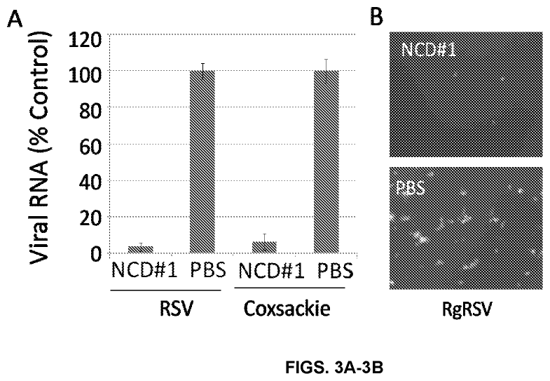

A culture supernatant was inoculated with either RSV or coxsackievirus and then was incubated with NCD1 at room temperature for 30 min and subjected to centrifugation to pellet the NCD1 complexes and RNA was isolated from supernatant after incubation. The RNA from supernatant containing virus and treated with PBS served as control. Results in FIG. 3A show that NCD1 was able to remove both RSV and coxsackievirus from the culture supernatant to the extent of 95-99%. The total RNA was isolated from the pellets representing complexes of NCD1 and RSV, as shown in FIG. 3B. The results show that the viral RNA was recovered from the pellet.

### Example 5. TPP-NCD1 Enables Neutralization of SARS-CoV-2

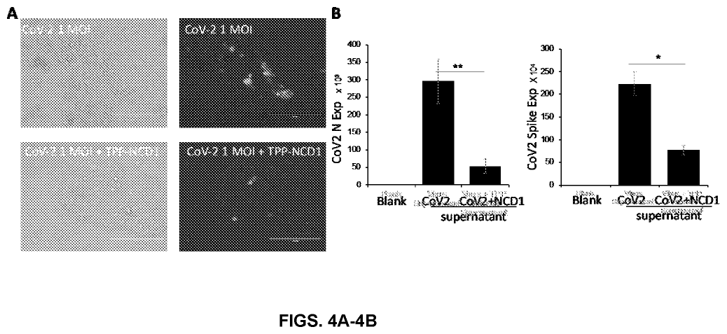

To test whether NCD1 enables neutralization of SARS-CoV-2, tri-polyphosphate (TPP) cross-linked NCD1 in Calu3 cells, which are susceptible to SARS-CoV2 virus, were used. Briefly, Calu3 cells were plated (300, 000/well) in a 6 well plate. One day after, Calu3 cells were infected with either 1 MOI SARS CoV2 virus or with 1 MOI SARS CoV2 virus preincubated (for 30) mins with 10 ug/ml TPP-NCD1. Forty-eight hours post infection the viral supernatant was collected and used to reinfect a fresh batch of Calu3 cells. After a further 48 hrs post infection, cells were examined for infection by fluorescent microscopy and the RNA was examined for SARS-CoV2 Spike and N gene expression by qPCR. The results showed that the SARS-CoV-2 replicated in Calu-3 cells. However, the presence of TPP-NCD1 inhibited their release to the supernatant (FIG. 4).

### Example 6. Shows Anti-HIV Activity in PBMC Culture Ex Vivo

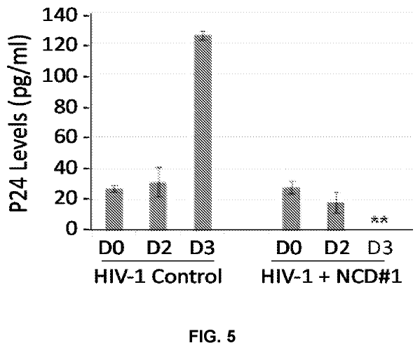

To confirm the activity of the newly synthesized compounds in HIV infected human cells, peripheral blood mononuclear cells (PBMCs) from healthy humans were cultured and parallel assays with PBMCs treated with NCD1 and samples with no treatment were run, followed by infection with HIV-1 89.6. Samples were pelleted on days 0, 2, and 3. Culture supernatants were assayed for p24 concentration. The results show that p24 levels were significantly reduced in NCD1-treated vs. non-treated cells on day 3 of the culture (FIG. 5).

### Example 7. NCD1 Analogs Tested for ANTI-HIV ACTIVITY in HIV Culture Supernatants In Vitro

To identify the compounds that bind HIV, the HIV containing supernatant from HIV-infected cells was incubated with the desired compound and neutralization of HIV (1 milligram of the compound and 35 pg (picograms) of HIV-89.6 was determined by analysis of the total HIV RNA in culture supernatant. Thus, the binding of polysaccharide compounds with HIV is examined in culture supernatants. The control (IM AA con) used is equal amount of IM acetic acid in a total volume of 100 μl with phosphate buffered saline (PBS). 10 mg/ml stock solution of NCD1 is prepared in water. The stock solution is diluted in PBS such that 1 mg of NCD1 is taken in 100 μl volume. Test compounds (NCD3, NCD4, NCD5, NCD6, NCD7, NCD8, NCD9) added at 1 mg of the compound and resuspended using 10% DMSO in PBS so that the total volume is 100 μ1. Three of the compounds NCD5, NCD6, NCD9 were insoluble in water. Similarly, 10% DMSO control contained equal amount of DMSO without any compound being added.

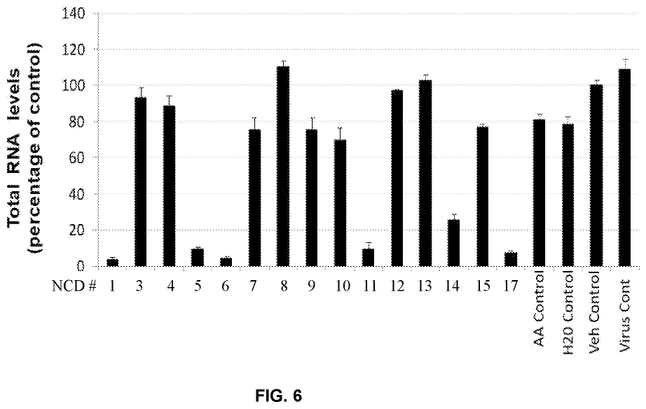

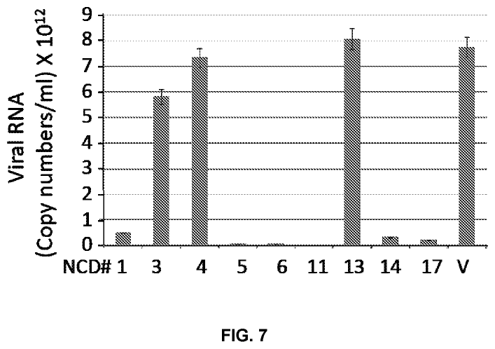

The results of RNA concentration in the supernatant showed that in addition to NCD1, several of its analogs such as NCD5, NCD6, NCD11, NCD14 and NCD17 showed more than 90% reduction in levels of HIV RNA in the supernatant. The rest of NCD1 analogs including NCD3, NCD4, NCD7, NCD8, NCD9, NCD10, NCD12, NCD13 and NCD15 did show less than 20% binding or in some cases, there was not binding to the virus at all (FIG. 6). The RNA copy numbers are shown in FIG. 7.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

1. A method for treating or preventing a viral infection or symptoms thereof in a subject in need thereof, the method comprising administering to the subject a therapeutically effective amount of an oligochitosan or a derivative thereof or a pharmaceutically acceptable salt or ester thereof having a molecular weight of at least about 1 KDa and a degree of deacetylation of at least about 1%.

2. The method of claim 1, wherein the oligochitosan or a derivative thereof has a molecular weight of from about 1 KDa to about 100 KDa.

3. The method of claim 1, wherein the oligochitosan or a derivative thereof has a degree of deacetylation of from about 10% to about 100%.

4. The method of claim 1, wherein the subject is administered oligochitosan having a molecular weight of from about 5 KDa to about 15 KDa and a degree of deacetylation of from about 10% to about 20%.

5. The method of claim 1, wherein the derivative of the oligochitosan comprises one or more glucosamine units having the structure I 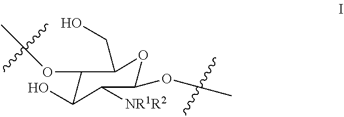
wherein R1 and R2 are independently an alkyl group, an aryl group, a carboxyl group having the formula C(O)X, where X is an alkyl group, an aryl group, or an alkoxy group, wherein both R1 and R2 are not hydrogen.

6. The method of claim 5, wherein R1 is hydrogen and R2 is C(O)X, where X is an alkylthiol group.

7. The method of claim 5, wherein R1 is hydrogen and R2 is C(O)X, where X is —(CH2)nSH, where n is an integer from 1 to 10.

8. The method of claim 5, wherein R1 is hydrogen and R2 is C(O)X, where X is an alkylcarboxylic acid group or an ester or salt thereof.

9. The method of claim 5, wherein R1 is hydrogen and R2 is C(O)X, where X is (CH2)oCO2H or the ester or salt thereof, where o is an integer from 1 to 10.

10. The method of claim 5, wherein R1 is hydrogen and R2 is C(O)X, where X is an alkyl hydroxy group with one or more carboxylic acid groups.

11. The method of claim 5, wherein R1 is hydrogen and R2 is C(O)X, where X is an alkyl hydroxy group with two carboxylic acid groups.

12. The method of claim 5, wherein the derivative of the oligochitosan comprises from about 10% to about 100% glucosamine units having the structure I.

13. The method of claim 1, wherein the derivative of the oligochitosan comprises one or more glucosamine units having the structure II 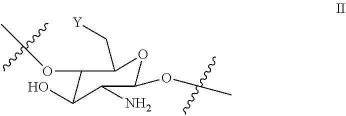
wherein Y is a halo group, an azide group, an alkyl group, an alkenyl group, an alkynyl group, or a carboxyl group having the formula C(O)X, where X is an alkyl group, an aryl group, an arylalkoxy group, a hydroxyl group, an alkoxy group, or a substituted or unsubstituted amine group.

14. The method of claim 13, wherein Y is a bromide group, an azide group, a propargyl group, or an aryloxy group comprising one or more phenyl groups.

15.-17. (canceled)

18. The method of claim 13, wherein Y is C(O)X, where X is a hydroxyl group.

19. The method of claim 13, wherein Y is C(O)X, where X is —NH(CH2)pNH2 and p is an integer from 1 to 10.

20. The method of claim 13, wherein the derivative of the oligochitosan comprises from about 10% to about 100% glucosamine units having the structure II.

21. The method of claim 1, wherein the oligochitosan or the derivative thereof is crosslinked.

22.-25. (canceled)

26. The method of claim 1, wherein the oligochitosan or the derivative thereof is administered as a pharmaceutical composition.

27.-36. (canceled)

37. A medical device comprising a coating of an oligochitosan or a derivative thereof having a molecular weight of at least 1 KDa and a degree of deacetylation of at least 1%.

38. (canceled)
